(19) United States
(12) Patent Application Publication
(10) Pub. No.: US 2025/0152852 A1
(43) Pub. Date: May 15, 2025

(54) SAFE INJECTION SYSTEMS AND METHODS

(71) Applicant: CREDENCE MEDSYSTEMS, INC., Menlo Park, CA (US)

(72) Inventors: Jeff Tillack, Wake Forest, NC (US); Everett Matthew Notaro, Santa Clara, CA (US); Alan E. Shluzas, San Carlos, CA (US)

(73) Assignee: CREDENCE MEDSYSTEMS, INC., Menlo Park, CA (US)

(21) Appl. No.: 18/946,512

(22) Filed: Nov. 13, 2024

Related U.S. Application Data
(60) Provisional application No. 63/598,375, filed on Nov. 13, 2023.

(51) Int. Cl. A61M 5/32; A61M 5/20

(52) U.S. Cl. CPC A61M 5/3257; A61M 5/20

(57) ABSTRACT

A system for injection includes a syringe body having proximal and distal ends, and defining a syringe body interior therebetween. The system also includes a needle hub assembly coupled to the syringe body distal end. The needle hub assembly includes a needle hub, and a needle removably coupled thereto and having a sharp needle distal end and a needle proximal end feature. The system further includes a stopper member movably disposed in the syringe interior. Moreover, the system includes a plunger member having proximal and distal ends, and coupled to the stopper member at the distal end. The plunger member is configured to insert the stopper member distally in the syringe interior. When the stopper member is inserted to the syringe body distal end, the plunger member proximal end is flush with the syringe body proximal end or disposed in the syringe body interior.

The present application claims priority to U.S. Provisional Patent Application Ser. No. 63/598,375, filed on Nov. 13, 2023 under attorney docket number CM.30044.00 and entitled “SAFE INJECTION SYSTEMS AND METHODS.” This application includes subject matter similar to the subject matter described in the following co-owned U.S. patent applications: (1) U.S. patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”; and (16) U.S. Provisional Patent Application Ser. No. 63/537,641, filed on Sep. 11, 2023 under attorney docket number CM.30037.01 and entitled “SAFE INJECTION SYSTEMS AND METHODS”. The contents of the applications and patents identified herein are fully incorporated herein by reference as though set forth in full.

### FIELD OF THE DISCLOSURE

The present disclosure relates generally to injection systems, devices, and processes for facilitating various levels of control over injection processes, and more particularly to devices and methods related to safe injection systems, devices, and processes that retract a sharp needle distal end within a needle hub after injection.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of-causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus, certain suppliers have moved to more “vertical” solutions, such as that (41) featured in FIG. 50, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Some safe injection systems such as those described in U.S. Pat. Nos. 10,010,677 and 10,926,038, which were previously incorporated by reference herein, include an autoinjector or a pen injector. Some plunger member thumb-caps may allow a user of an autoinjector or pen injector to remove the plunger member and the needle coupled thereto after injection from the safe injection system, thereby presenting a risk of unintended needle sticks. Accordingly, there exists a need for safe injection systems with plunger members that address these limitations.

### SUMMARY

Embodiments are directed to injection systems. In particular, the embodiments are directed to safe injection systems.

According to one embodiment, a system for injection includes a syringe body having a syringe body proximal end and a syringe body distal end, and defining a syringe body interior therebetween. The system also includes a needle hub assembly coupled to the syringe body at the syringe body distal end. The needle hub assembly includes a needle hub, and a needle removably coupled to the needle hub and having a sharp needle distal end and a needle proximal end feature. The system further includes a stopper member movably disposed in the syringe interior. Moreover, the system includes a plunger member having a plunger member proximal end and a plunger member distal end, and coupled to the stopper member at the plunger member distal end. The plunger member is configured to insert the stopper member distally in the syringe interior relative to the syringe body and the needle hub. The plunger member and the syringe body are configured such that, when the stopper member is inserted to the syringe body distal end, the plunger member proximal end is flush with the syringe body proximal end or disposed in the syringe body interior.

In one or more embodiments, the plunger member includes a plunger member body defining a plunger interior, a needle retention member disposed in the plunger interior and configured to couple to the needle proximal end feature, an energy-storage member disposed in the plunger interior and configured to withdraw the needle retention member proximally in the plunger interior, and an energy-storage member latching member disposed in the plunger interior. The energy-storage member may have a latched state in which the energy-storage member latching member holds the energy-storage member in an energized state, and an unlatched state in which the energy-storage member latching member does not restrain the energy-storage member and the energy-storage member transforms into a released state. When the stopper member is inserted to the syringe body distal end, the energy-storage member latching member may transform from the latched state to the unlatched state, and the energy-storage member may pull needle proximally such that the sharp needle distal end is disposed in an interior of the needle hub.

In one or more embodiments, when the stopper member is inserted to the syringe body distal end, the energy-storage member latching member is transformed from the latched state to the unlatched state, and the energy-storage member pulls the needle proximally via the needle retention feature coupled to the needle proximal end feature. The plunger member may also include a plunger cap configured to close the plunger member proximal end to contain the needle at least partially in the plunger interior when the energy-storage member pulls needle proximally. The plunger member proximal end may include a hinge configured to facilitate coupling the plunger cap to the plunger member.

In one or more embodiments, the needle proximal end feature is configured pierce partially through and be disposed in the stopper member, and the needle proximal end feature and the stopper member are configured to form a fluid-tight seal when the needle proximal end feature is pierced partially through and disposed in the stopper member. The needle proximal end feature may include a cylindrical portion configured to form a fluid-tight seal with the stopper member. The stopper member may be made from an elastic polymer, and the cylindrical portion of the needle proximal end feature may be made from a metal.

In one or more embodiments, the syringe body proximal end defines a syringe body inner diameter, and the plunger member proximal end defines a plunger member outer diameter less than the syringe body inner diameter such that the plunger member proximal end can be inserted into the syringe body interior. The syringe body and the plunger member may be configured to prevent manual removal of the plunger member from the syringe body interior after the plunger member proximal end is inserted into the syringe body interior.

According to another embodiment, a system for injection includes a syringe body having a syringe body proximal end and a syringe body distal end, and defining a syringe body interior therebetween. The system also includes a needle hub assembly coupled to the syringe body at the syringe body distal end. The needle hub assembly includes a needle hub, and a needle removably coupled to the needle hub and having a sharp needle distal end and a needle proximal end feature. The system further includes a stopper member movably disposed in the syringe interior. Moreover, the system includes a plunger member having a plunger member proximal end and a plunger member distal end. In addition, the system includes a needle retention member releasably coupled to the plunger member and configured to couple to the needle proximal end feature. The plunger member is configured to insert the stopper member distally in the syringe interior relative to the syringe body and the needle hub. The plunger member and the syringe body are configured such that, when the stopper member is inserted to the syringe body distal end, the needle proximal end pierces the stopper member to couple to the needle retention member and the needle retention member proximal end is disposed in the syringe body interior.

In one or more embodiments, the plunger member includes a plunger member body defining a plunger interior. The plunger member also includes the needle retention member disposed in the plunger interior. The plunger member further includes an energy-storage member disposed in the plunger interior and configured to withdraw the needle retention member proximally in the plunger interior. Moreover, the plunger member includes an energy-storage member latching member disposed in the plunger interior. The energy-storage member latching member has a latched state in which the energy-storage member latching member holds the energy-storage member in an energized state, and an unlatched state in which the energy-storage member latching member does not restrain the energy-storage member and the energy-storage member transforms into a released state. When the stopper member is inserted to the syringe body distal end, the energy-storage member latching member is transformed from the latched state to the unlatched state, and the energy-storage member pulls needle proximally such that the sharp needle distal end is disposed in an interior of the needle hub.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

FIGS. 1A to 5C illustrate various aspects of conventional injection syringe configurations.

FIG. 6 is a perspective view of a safe injection system according to some embodiments.

FIG. 7 is a longitudinal cross-sectional view of a safe injection system before injection according to some embodiments.

FIG. 8A is a longitudinal cross-sectional view of a safe injection system after injection and needle retraction according to some embodiments.

FIG. 8B is a detailed longitudinal cross-sectional view of the distal end of a safe injection system after injection and needle retraction according to some embodiments.

FIG. 9 is an exploded view of a needle assembly according to some embodiments.

FIG. 10 is a detailed exploded perspective view of the proximal end of a safe injection system according to some embodiments.

FIGS. 11 to 15 are longitudinal cross-sectional views depicting a method of using a safe injection system to perform an injection and to retract a needle after the injection for safe disposal of the used system according to some embodiments.

FIG. 16 is a detailed longitudinal cross-sectional view of the distal end of a safe injection system after injection and needle retraction according to some embodiments.

FIG. 17 is a detailed longitudinal cross-sectional view of the proximal end of a safe injection system after injection and needle retraction according to some embodiments.

FIGS. 18 to 25 are isometric views depicting a method of using a safe injection system in an autoinjector, according to some embodiments.

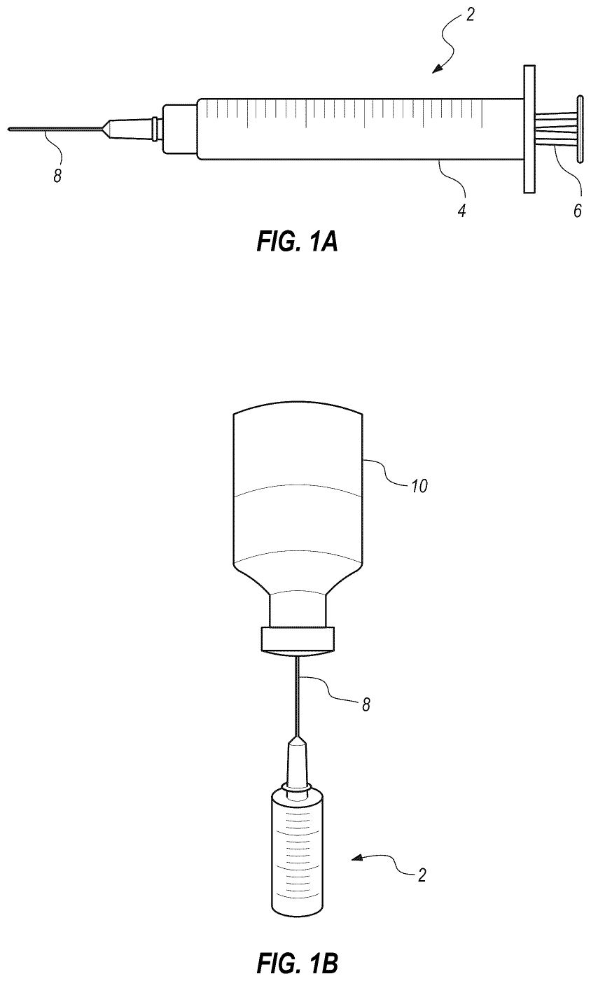

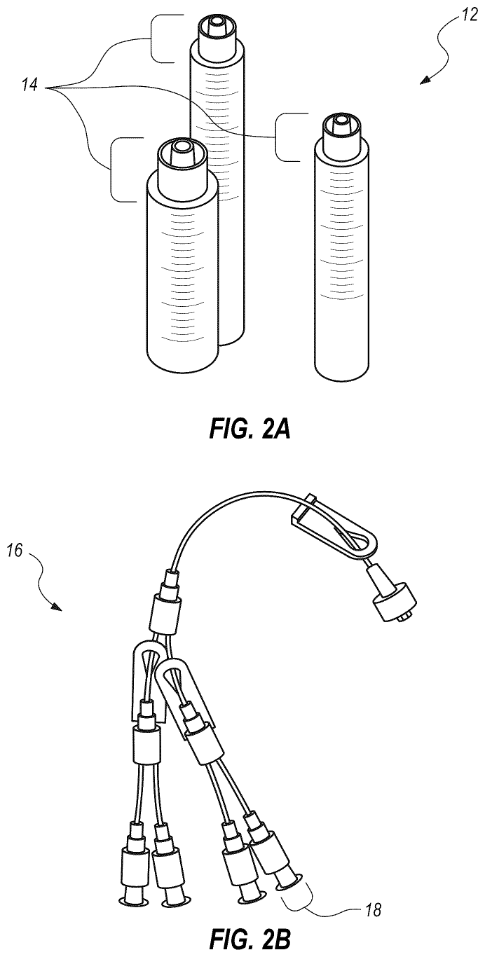

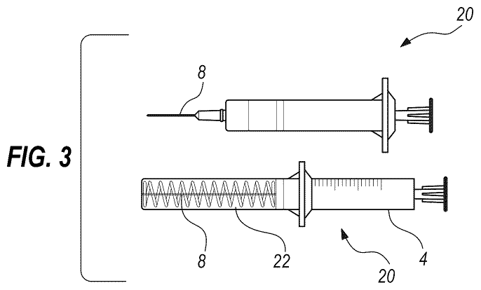

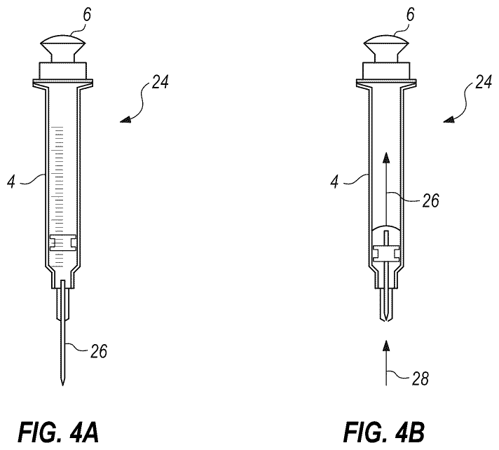

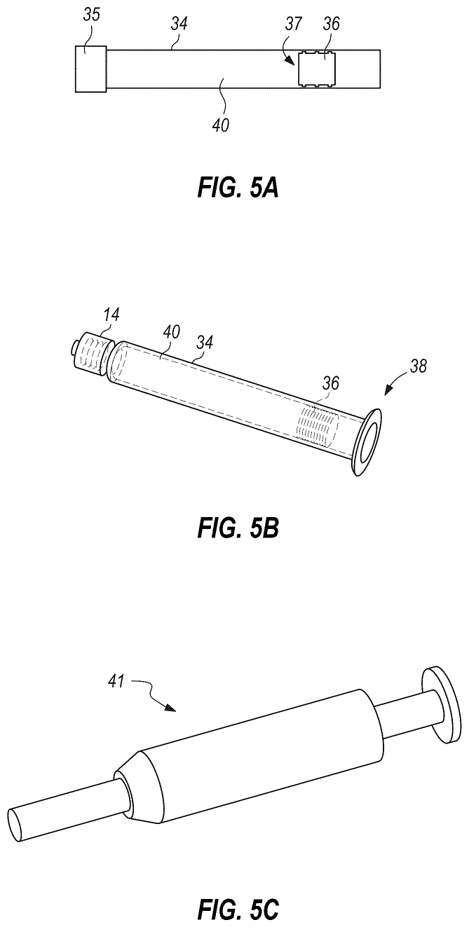

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Safe Injection System

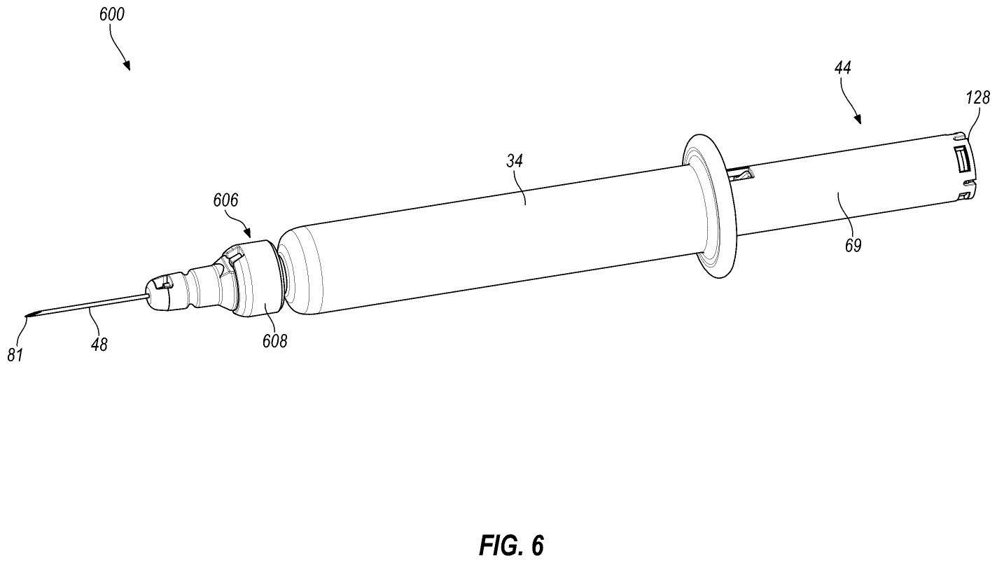

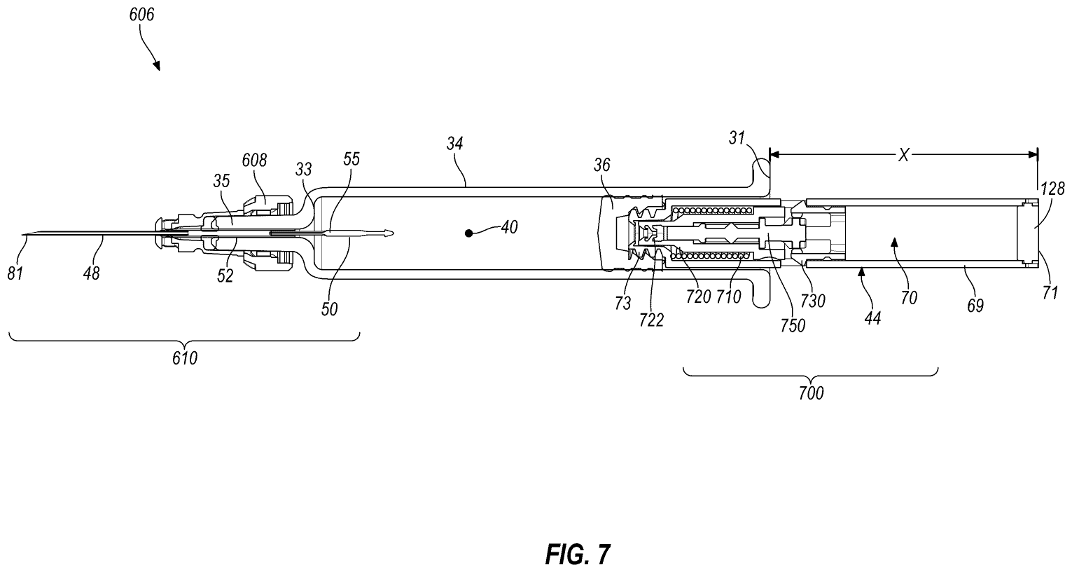

Referring to FIGS. 6 and 7, perspective and longitudinal cross-sectional views of a safe injection system (600) before injection are shown, with a conventional off-the-shelf pre-filled syringe body (34) with a conventional off-the-shelf stopper member (36) disposed therein according to some embodiments. The syringe body (34) defines a syringe body proximal end (31) and a syringe body distal end (33). The stopper member (36) together with the syringe body (34) define a syringe chamber (40). The stopper member (36) occludes the proximal end (31) of the syringe chamber (40). The safe injection system (600) also includes a plunger member (44) coupled to a proximal end of the stopper member (36), for instance with a conventional screw-on connection. The plunger member (44) defines a plunger member proximal end (71) and a plunger member distal end (73). A needle hub assembly (606) is disposed at the distal end of the syringe chamber (40).

The safe injection system (600) includes a needle retraction system (700) for use with safe injection systems such as the system (600) depicted in FIG. 7 according to some embodiments. The needle retraction system (700) includes an energy-storage member/spring (710) shown in an energized/compressed state, a needle catch (720), and a trigger (750). Most of the components of the needle retraction system (700) are disposed in the plunger interior (70) defined by the plunger body (69). As such, the plunger member (44) has approximately the same dimensions as a conventional plunger member and is usable with the conventional syringe body (34) and conventional stopper member (36).

The needle catch (720) defines a pair of rotatable energy-storage member latches (730) configured to interfere with the plunger body (69) to hold the energy-storage member (710) in the energized state depicted in FIG. 7. The needle catch (720) also defines a pair of rotatable needle latch arms (722) configured to interfere with the radially expanded member (56) on the needle proximal member (50) to allow the radially expanded member (56) to move proximally past the rotatable needle latch arms (722) while preventing the radially expanded member (56) from moving distally past the plurality of rotatable needle latch arms (722). As such, the needle latch arms (722) allow the radially expanded member (56) and the needle proximal member (50) to be caught or captured by the needle catch (720).

The safe injection system (600) controls injection of a fluid from the syringe chamber (40) through the needle hub assembly (606) driven by distal insertion of the plunger member (44) relative to the syringe body (34). This distal insertion of the plunger member (44) relative to the syringe body (34) may be performed automatically by an autoinjector or manually by a user. The plunger member (44) includes a plunger housing member (69) defining a plunger interior (70) and a plunger cap (128) coupled to the plunger housing member (69) at a proximal end (31) thereof.

The safe injection system (600) has a staked needle configuration wherein upon presentation to the user, a needle hub assembly (606), comprising a needle hub (608) and a needle assembly (610), is mounted in position ready for injection. The distal tip of the syringe body (34) comprises a luer taper interface (35) configured to facilitate coupling of the needle hub assembly (606) to the syringe body (34).

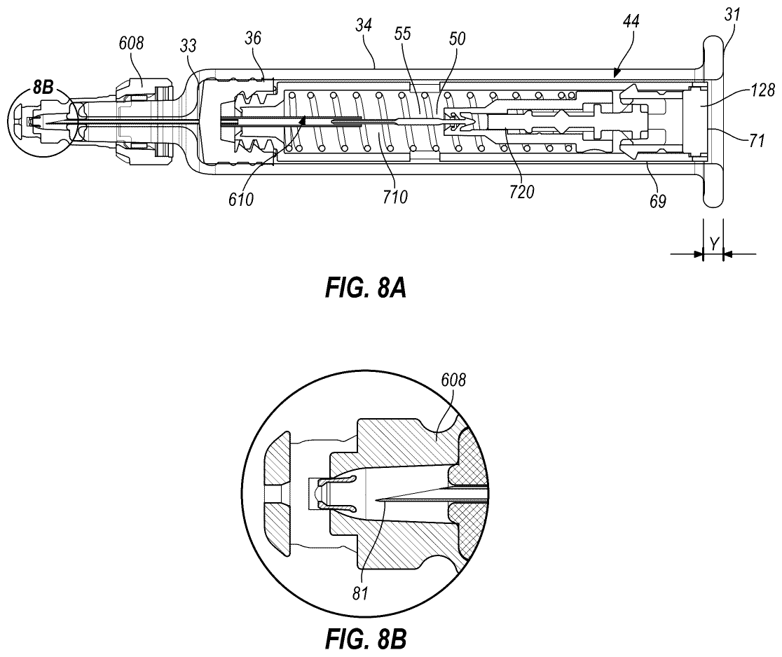

FIGS. 8A and 8B are longitudinal cross-sectional and detailed longitudinal cross-sectional views of a safe injection system (600) after injection and needle retraction according to some embodiments.

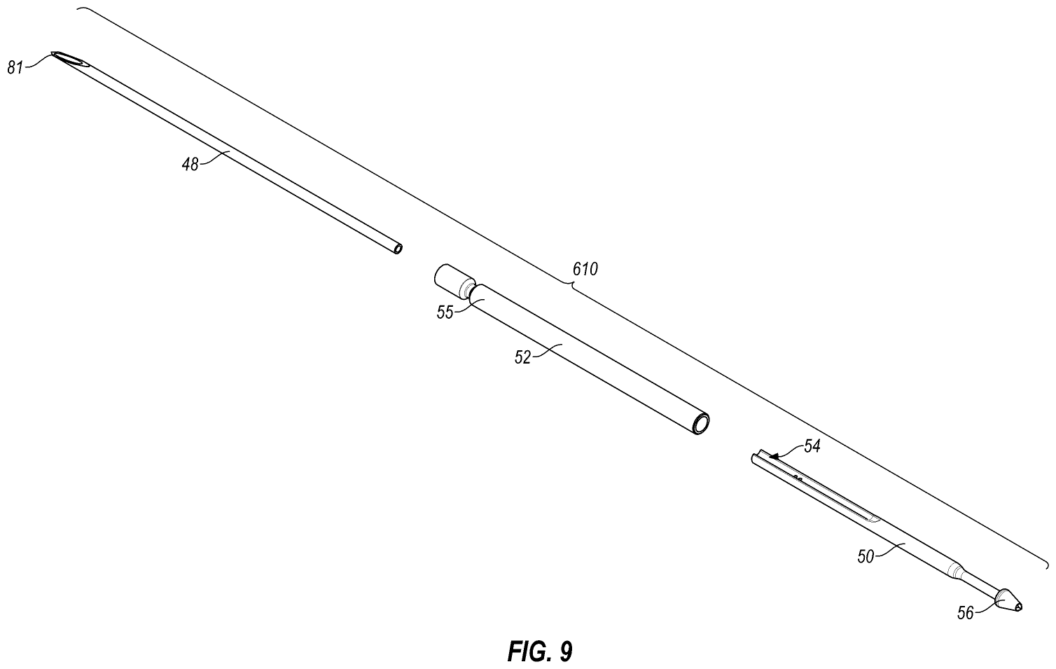

FIG. 9 is an exploded view of the needle assembly (610) according to some embodiments. The needle assembly (610) includes a needle distal member (48) and a needle proximal member (50) coupled to each other by a needle joining member (52). The needle distal member (48) defines a sharp needle distal end (81). The needle proximal member (50) defines a radially expanded member (56) at a proximal end thereof and a cylindrical portion (55). A distal end of the needle proximal member (50) defines a pair of longitudinal channels (54), which form a fluid path from the syringe chamber (40), through the longitudinal channels (54), through the needle joining member (52), through the needle distal member (48), and to an exterior of the safe injection system (600) (see FIG. 6). While a pair of longitudinal channels (54) is shown, an alternative embodiment may be one longitudinal channel (54) formed in the needle proximal member (50). Additionally, three or more longitudinal channels (54) may be formed in the needle proximal end. (50).

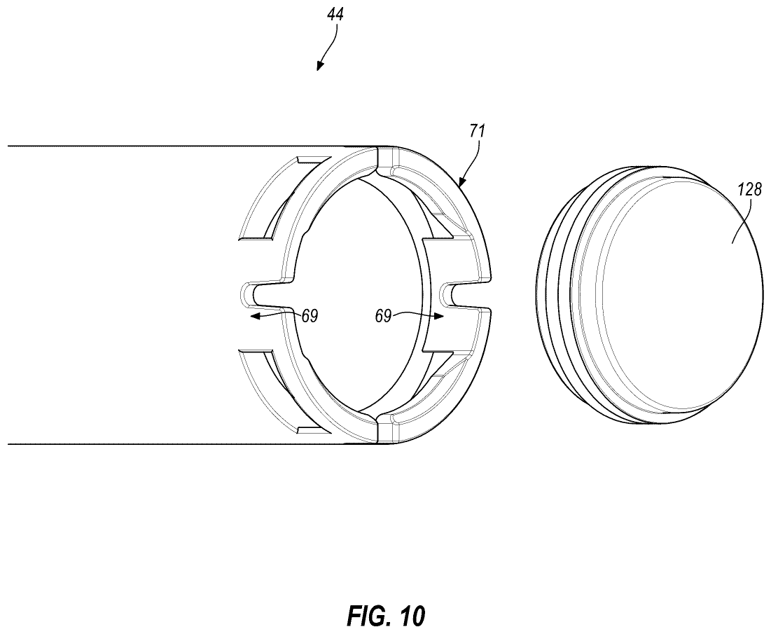

FIG. 10 is an exploded view of a plunger member proximal end (71) according to some embodiments. The proximal end (71) of the plunger member (44) defines a pair of hinges (69) configured to allow the proximal end (71) to open to allow the plunger cap (128) to be disposed therein.

While the staked needle is depicted as mounted in position, the staked needle may be removably coupled to the syringe body (34) using a Luer interface (not shown), with the proximal member (50) of the needle member extending through the Luer interface and into the syringe chamber (40). Alternatively, the needle may be fixedly or removably mounted to the flange on a cartridge body instead of a syringe. Such cartridge injection systems are disclosed in U.S. patent application Ser. No. 15/801,281, which was previously incorporated by reference herein. Alternatively, the needle may be fixedly or removably mounted to the tip of a dual chamber injection system. Such dual chamber injection systems are disclosed in U.S. patent application Ser. No. 15/801,259, which was previously incorporated by reference herein. Alternatively, the needle may be fixedly or removably mounted to the tip of a sequential injection system. Such dual chamber injection systems are disclosed in U.S. patent application Ser. No. 17/364,546, which was previously incorporated by reference herein. In the embodiment depicted in FIG. 6, a significant portion of the safe needle retraction hardware resides in the plunger interior (70) defined by the plunger housing (69).

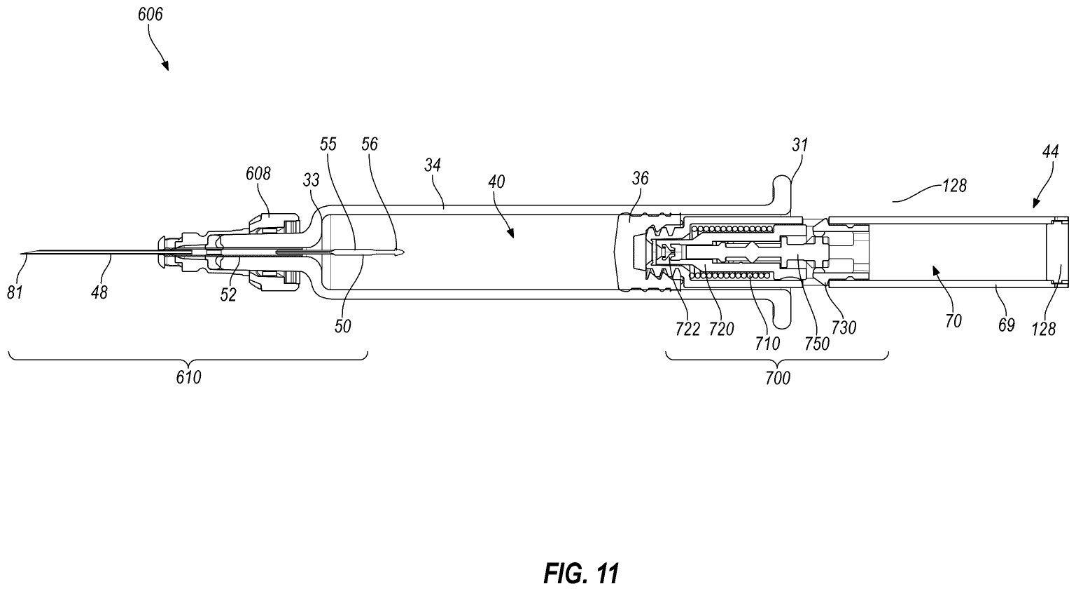

FIGS. 11 to 15 are longitudinal cross-sectional views depicting a method of using the safe injection system (600) depicted herein to perform an injection and to retract a needle (610) after the injection for safe disposal of the used system (600) according to some embodiments.

FIG. 11 depicts the safe injection system (600) in a ready for injection state. The syringe chamber (40) defined by the syringe body (34) is prefilled with an injectable fluid (not shown). The sharp distal end (81) of the distal needle member (48) may be protected by the needle cover (not shown) in a transport/storage state. The energy storage member (710) is held in a compressed state in the plunger interior (70) by interference between the energy-storage member latches (730) and the plunger body (69).

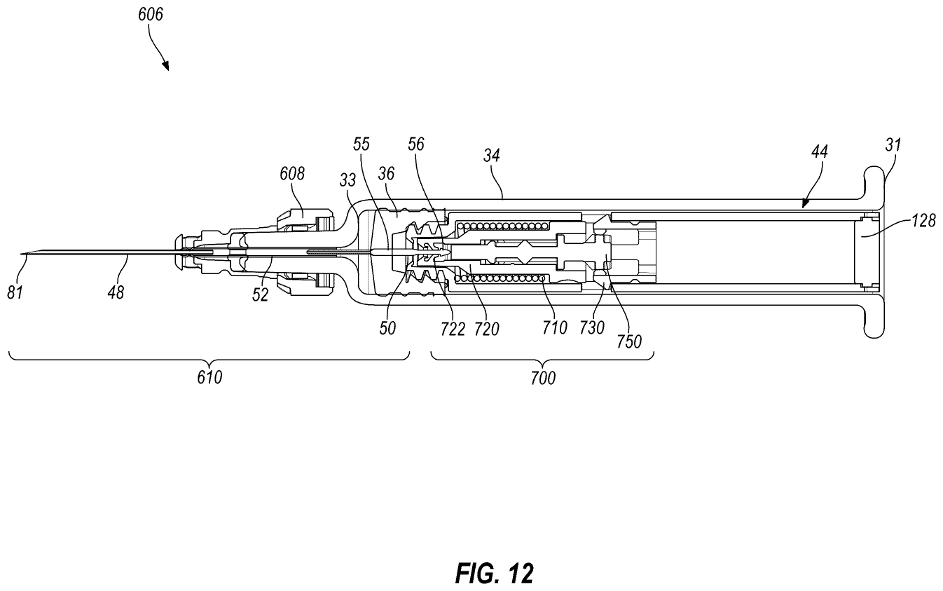

FIG. 12 depicts the safe injection system (600) after a distally directed force has been applied to the stopper member (36) through the plunger member (44), for instance by an autoinjector or a user exerting a distally directed force on a plunger cap (128). The radially expanded member (56) of the needle proximal member (50) has penetrated through the stopper member (36) and the needle catch (720) proximally pass the needle latches (722), effectively coupling the needle assembly (610) to the needle catch (720). The energy storage member (710) remains in a compressed state in the plunger interior (70). Because the needle proximal member (50) has yet to move the trigger (750) proximally, the energy storage member (710) remains in a compressed state in the plunger interior (70).

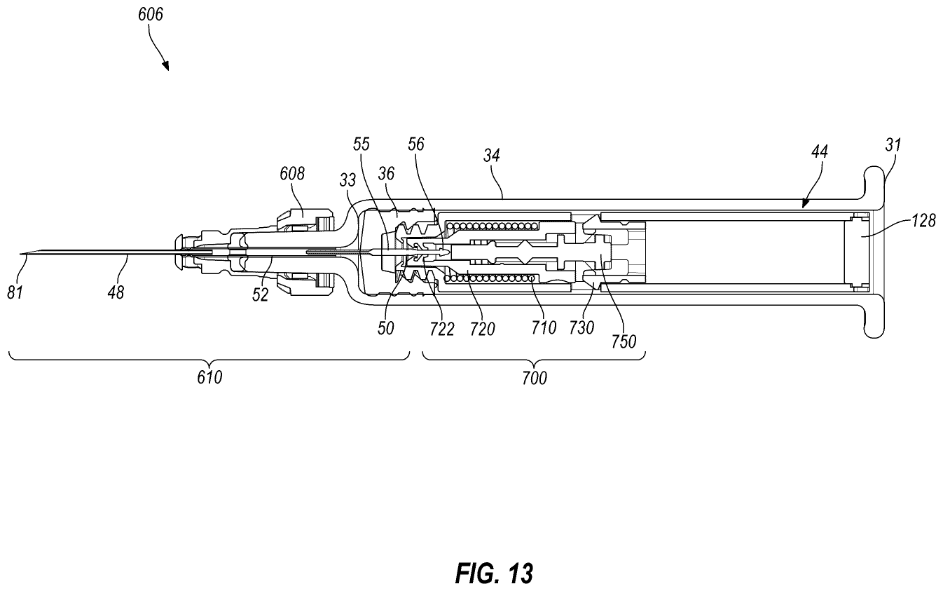

FIG. 13 depicts the safe injection system (600) after further distally directed force has been applied to the stopper member (36) through the plunger member (44) thereby moving the stopper member (36) to the distal end (33) of the syringe body (34). The radially expanded member (56) of the needle proximal member (50) has moved further proximally pass the needle latches (722) in the needle catch (720), and has moved the trigger (750) proximally to allow the energy-storage member latches (730) to rotate inward away from the plunger body (69) to transform the energy-storage member latches (730) from a latched state to an unlatched state. This transformation of the energy-storage member latches (730) allows the energy storage member (710) to transform from an energized state/compressed to an unenergized/expanded state.

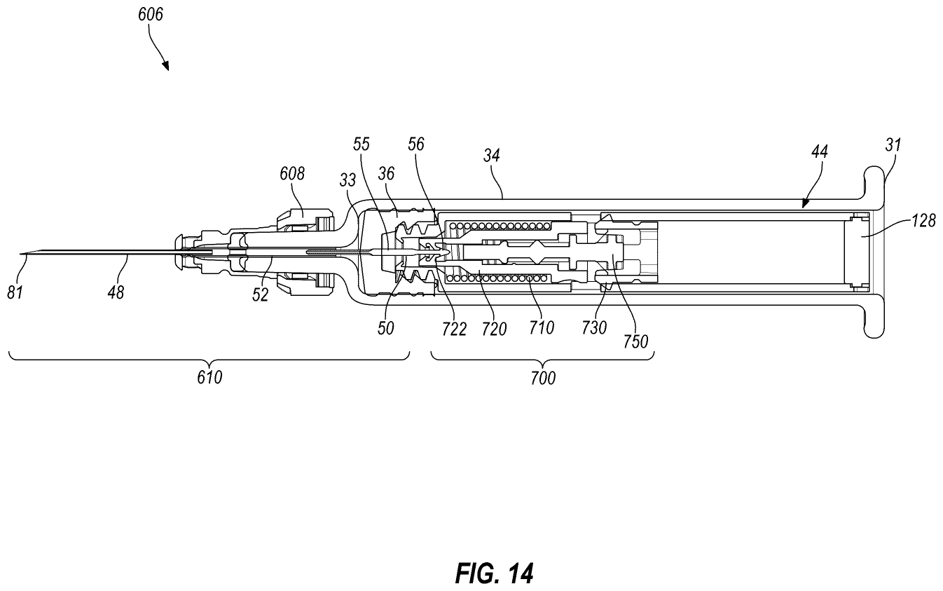

FIG. 14 depicts the safe injection system (600) after the energy-storage member (710) has partially expanded proximally to move the needle catch (720) and its needle latch arms (722) proximally such that the needle latch arms (722) engage the radially expanded member (56) of the needle proximal member (50) in a proximal direction.

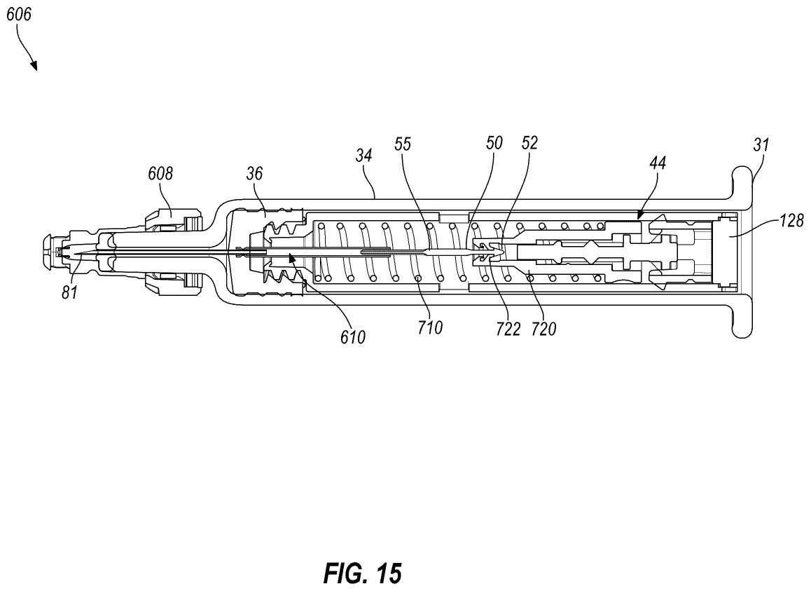

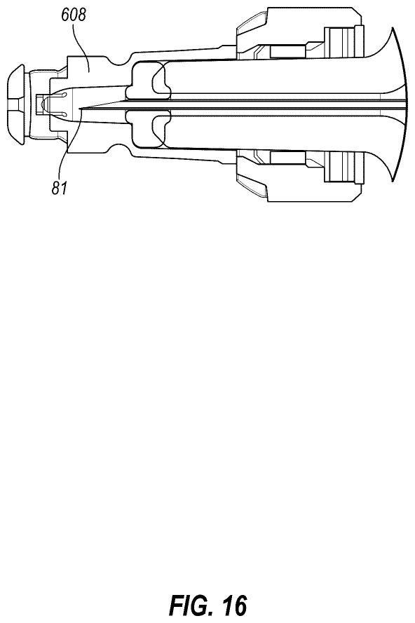

FIG. 15 depicts the safe injection system (600) after the released energy storage member (710) has fully expanded proximally, thereby moving the needle catch (720) proximally in the plunger interior (70) and retracting the needle (610) partially into the plunger interior (70). After the needle (610) is retracted, the sharp needle distal end (81) is disposed in the needle hub (608) rendering the system (600) safe for disposal while minimizing unintended needle sticks. FIGS. 8B and 16 depict the sharp needle distal end (81) disposed in the needle hub (608). Disposing of a safe injection system (600) after injection and needle retraction may include removing the safe injection system (600) from an autoinjector, thereby allowing the autoinjector to be reused in some embodiments.

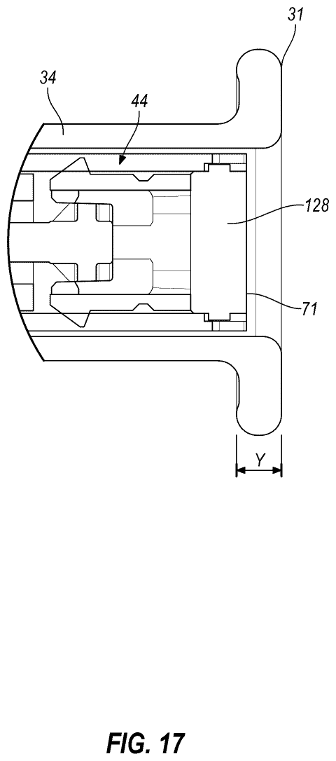

FIG. 17 depicts the proximal end of the safe injection system (600) after injection and needle retraction. The plunger member proximal end (71) defined by the plunger cap (128) is disposed in an interior of the syringe body (34) distal of the syringe body proximal end (31). The plunger member (44) has an outer diameter less than an inner diameter of the syringe body (34) to enable the plunger member proximal end (71) to be disposed in the interior of the syringe body (34). The plunger member proximal end (71) being disposed in the interior of the syringe body (34) renders the post injection and needle retraction safe injection system (600) “tamperproof,” in that it is very difficult for a user to manually remove the plunger member (44) and the needle (610) from the system (600). This minimizes the risk of unintended needle sticks.

In most embodiments, the plunger member proximal end (71) does not include a radially extending flange. While in some embodiments, a small radially extending flange may be included, this small radially extending renders the safe injection system less “tamperproof”.

The safe injection system (600) may be tuned for use with syringe bodies (34) of different sizes. In an embodiment with a 2.25 ml syringe body (34), a distance (X, see FIG. 7) between the syringe body proximal end (31) and the plunger member proximal end (71) before injection may be around 1.258 in. In such an embodiment, the syringe chamber (40) may contain around 2 ml of injectable fluid/medicine. Further, in such an embodiment, a distance (Y, see FIGS. 8A and 17) between the syringe body proximal end (31) and the plunger member proximal end (71) before injection may be around 0.064 in.

In another embodiment with a 1 ml syringe body (34), a distance (X, see FIG. 7) between the syringe body proximal end (31) and the plunger member proximal end (71) before injection may be around 1.150 in. In such an embodiment, the syringe chamber (40) may contain around 1 ml of injectable fluid/medicine. Further, in such an embodiment, a distance (Y, see FIGS. 8A and 17) between the syringe body proximal end (31) and the plunger member proximal end (71) before injection may be around 0.085 in.

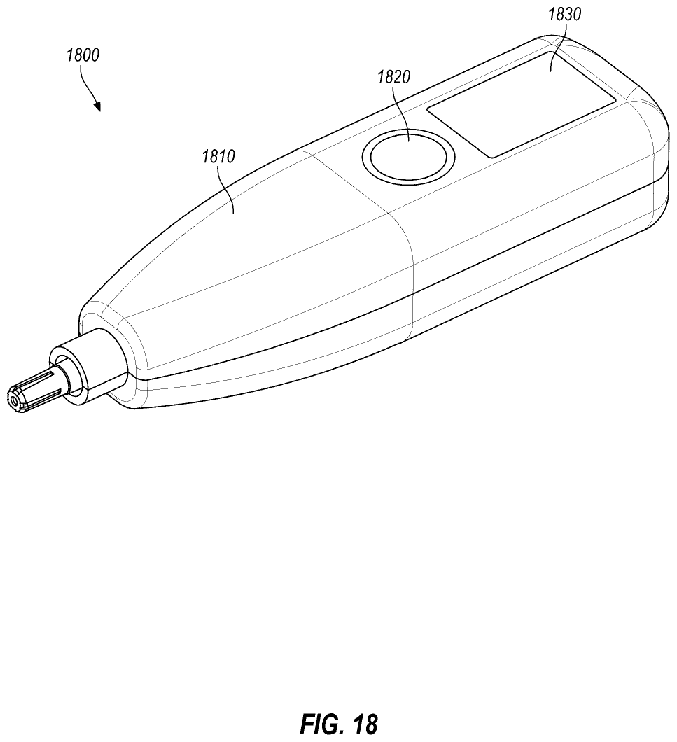

FIGS. 18 to 25 depict a method of using a safe injection system in an autoinjector according to some embodiments.

FIG. 18 depicts an autoinjector (1800) configured to perform an injection using a safe injection system (600), such as those described herein. The autoinjector (1800) comprises a housing (1810) configured to receive a prefilled safe injection system (600) therein. The autoinjector (1800) may be a reusable device, configured to accept a filled safe injection system (600), and to release the safe injection system (600) after the injection is given. Alternatively, the autoinjector (1800) may be configured to be disposed of with the safe injection system (600) after the injection is given. The autoinjector (1800) may include an activation button (1820) to initiate the injection sequence and/or a display (1830) to communicate with a user.

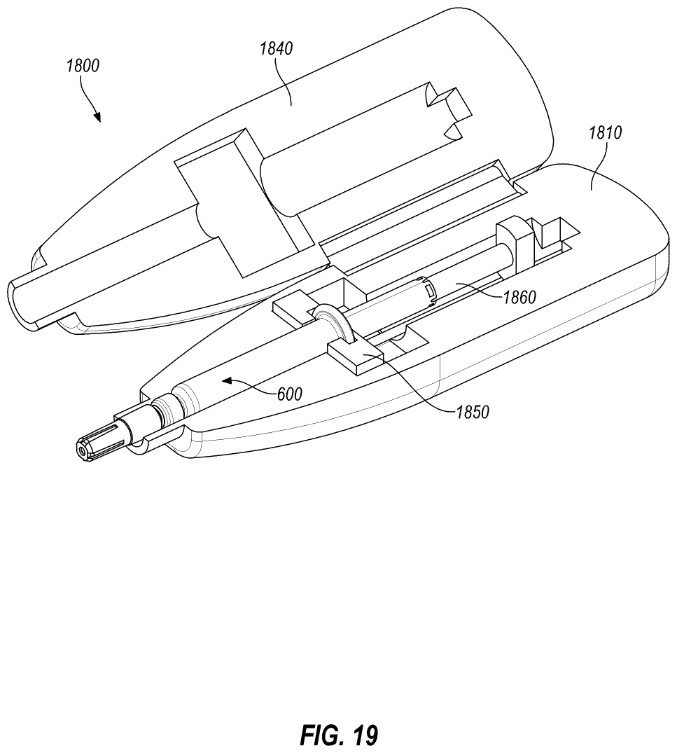

FIG. 19 depicts the autoinjector (1800) with a cover (1840) open for clarity. The autoinjector (1800) consists of a syringe nest (1850) configured to accept the safe injection system (600). The syringe nest (1850) is further configured to be advanced and/or retracted along the longitudinal axis of the autoinjector (1800) modulating the amount of needle projection from the end of the autoinjector (1800). The safe injection system (600) is housed in a cradle inside the autoinjector (1800). The autoinjector (1800) pushrod is configured to couple to the plunger rod of the safe injection system (600). An alternative embodiment of the autoinjector (1800) holds the safe injection system (600) in a fixed point along the length of the autoinjector (1800), allowing the user to advance the needle into the patient to perform the injection. The autoinjector (1800) further comprises a pushrod (1860) which is configured to be advanced distally and/or retracted proximally along the longitudinal axis of the autoinjector (1800) to control the position of the intercoupled plunger member and/or the stopper member inside the safe injection system (600). The position of the pushrod (1860) is controlled by a motor, or may be controlled by a spring.

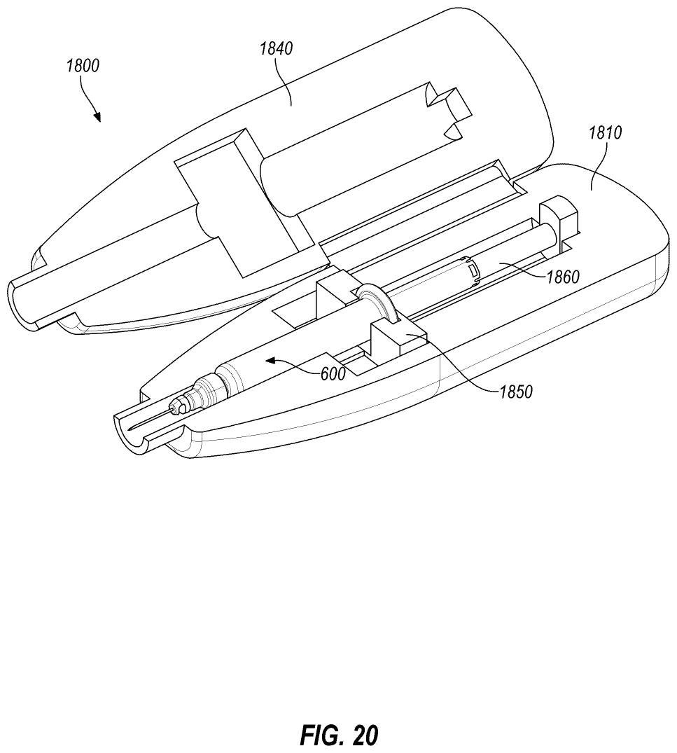

FIG. 20 depicts the autoinjector (1800) with the safe injection system (600) moved proximal to prepare for injection. The autoinjector (1800) may be configured to remove a needle shield at this step.

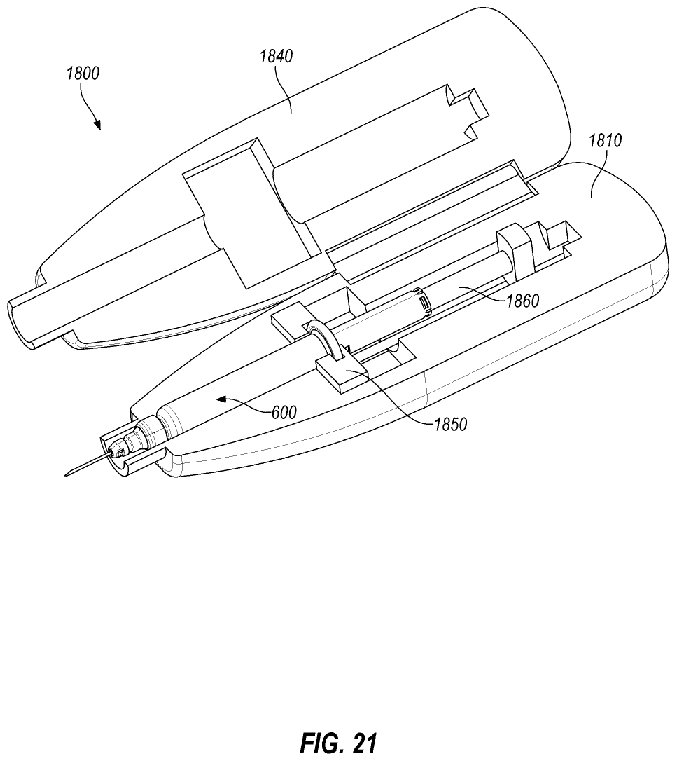

FIG. 21 depicts the autoinjector (1800) advancing the needle into a patient. The syringe nest (1850) is configured to move distally, advancing the safe injection system (600) and the intercoupled needle into the patient.

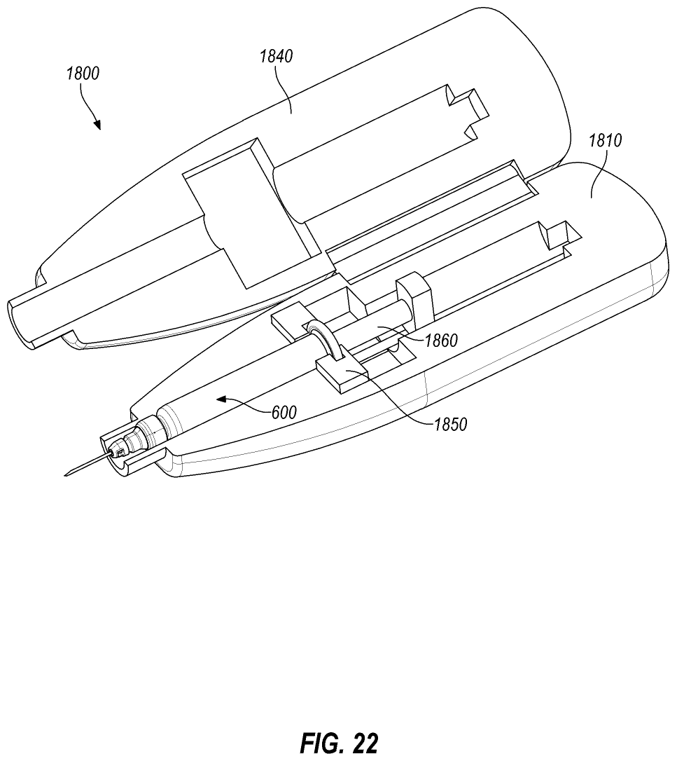

FIG. 22 depicts the push rod (1860) of the autoinjector (1800) advancing the plunger rod and the intercoupled stopper towards the distal end of the syringe body, expelling the medicine out of the needle into the patient.

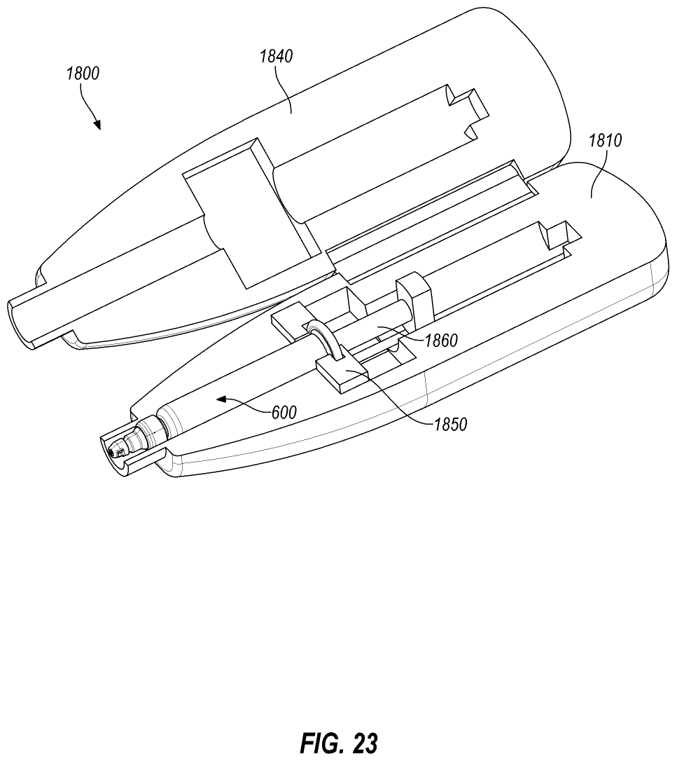

FIG. 23 depicts the needle-sharp end being retracted into the needle hub and rendered safe.

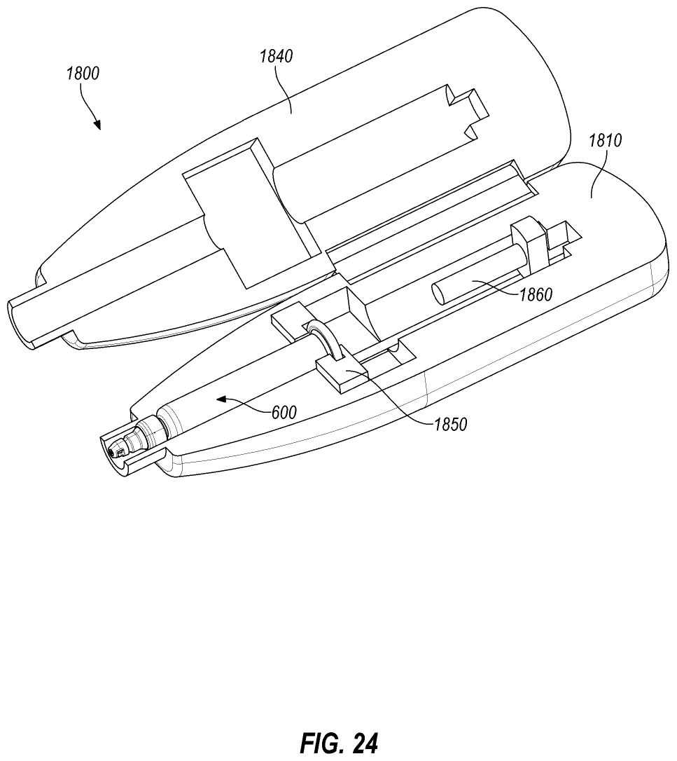

FIG. 24 depicts the autoinjector pushrod (1860) moving proximally and and/or decoupling from the plunger rod of the safe injection system (600).

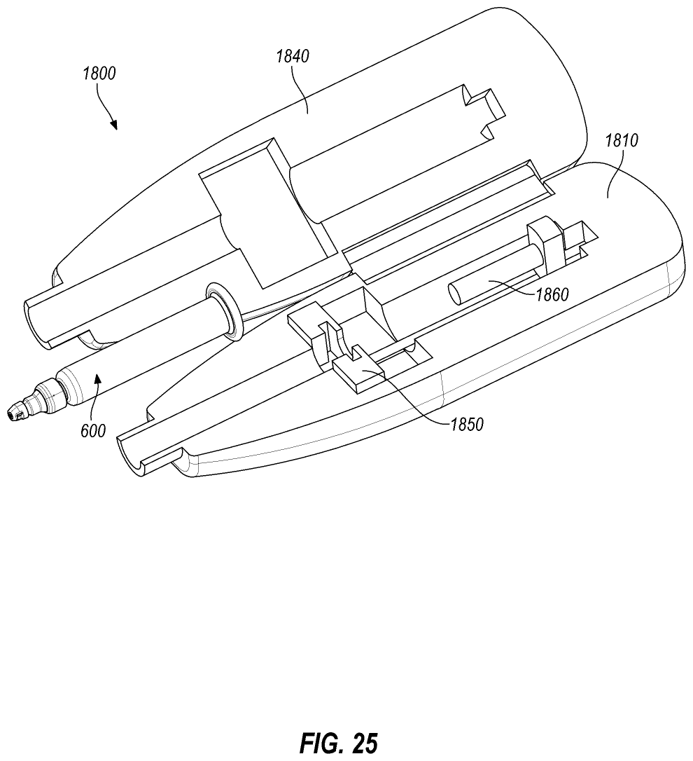

FIG. 25 depicts the empty safe injection system (600) being removed from the autoinjector (1800) for disposal.

While the embodiments described above include single chamber safe injection systems, the scope of the claims also include multiple chamber injection systems. For multiple chamber safety injection systems, two or more stopper members are inserted into an injection system body (e.g., syringe body, cartridge body, etc.) to define a corresponding number of chambers.

While the safe injection systems depicted and described herein include syringe bodies with staked needles, the safe injection systems described herein can include syringe bodies with Luer connectors, etc.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding

material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element--irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A system for injection, comprising:
a syringe body having a syringe body proximal end and a syringe body distal end, and defining a syringe body interior therebetween;
a needle hub assembly coupled to the syringe body at the syringe body distal end, and comprising
a needle hub, and
a needle removably coupled to the needle hub and having a sharp needle distal end and a needle proximal end feature;
a stopper member movably disposed in the syringe interior; and
a plunger member having a plunger member proximal end and a plunger member distal end, and coupled to the stopper member at the plunger member distal end,
wherein the plunger member is configured to insert the stopper member distally in the syringe interior relative to the syringe body and the needle hub, and
wherein the plunger member and the syringe body are configured such that, when the stopper member is inserted to the syringe body distal end, the plunger member proximal end is flush with the syringe body proximal end or disposed in the syringe body interior.

2. The system of claim 1, wherein the plunger member comprises
a plunger member body defining a plunger interior,
a needle retention member disposed in the plunger interior and configured to couple to the needle proximal end feature,
an energy-storage member disposed in the plunger interior and configured to withdraw the needle retention member proximally in the plunger interior, and
an energy-storage member latching member disposed in the plunger interior and having
a latched state in which the energy-storage member latching member holds the energy-storage member in an energized state, and
an unlatched state in which the energy-storage member latching member does not restrain the energy-storage member and the energy-storage member transforms into a released state, and
wherein when the stopper member is inserted to the syringe body distal end, the energy-storage member latching member is transformed from the latched state to the unlatched state, and the energy-storage member pulls needle proximally such that the sharp needle distal end is disposed in an interior of the needle hub.

3. The system of claim 2, wherein when the stopper member is inserted to the syringe body distal end, the energy-storage member latching member is transformed from the latched state to the unlatched state, and the energy-storage member pulls the needle proximally via the needle retention feature coupled to the needle proximal end feature.

4. The system of claim 2, wherein the plunger member further comprises a plunger cap configured to close the plunger member proximal end to contain the needle at least partially in the plunger interior when the energy-storage member pulls needle proximally.

5. The system of claim 4, wherein the plunger member proximal end comprises a hinge configured to facilitate coupling the plunger cap to the plunger member.

6. The system of claim 1, wherein the needle proximal end feature is configured pierce partially through and be disposed in the stopper member, and wherein the needle proximal end feature and the stopper member are configured to form a fluid-tight seal when the needle proximal end feature is pierced partially through and disposed in the stopper member.

7. The system of claim 6, wherein the needle proximal end feature comprises a cylindrical portion configured to form a fluid-tight seal with the stopper member.

8. The system of claim 7, wherein the stopper member is made from an elastic polymer, and
wherein the cylindrical portion of the needle proximal end feature is made from a metal.

9. The system of claim 1, wherein the syringe body proximal end defines a syringe body inner diameter, and
wherein the plunger member proximal end defines a plunger member outer diameter less than the syringe body inner diameter such that the plunger member proximal end can be inserted into the syringe body interior.

10. The system of claim 9, wherein the syringe body and the plunger member are configured to prevent manual removal of the plunger member from the syringe body interior after the plunger member proximal end is inserted into the syringe body interior.

11. A system for injection, comprising:
a syringe body having a syringe body proximal end and a syringe body distal end, and defining a syringe body interior therebetween;
a needle hub assembly coupled to the syringe body at the syringe body distal end, and comprising
a needle hub, and
a needle removably coupled to the needle hub and having a sharp needle distal end and a needle proximal end feature;
a stopper member movably disposed in the syringe interior;
a plunger member having a plunger member proximal end and a plunger member distal end; and
a needle retention member releasably coupled to the plunger member and configured to couple to the needle proximal end feature,
wherein the plunger member is configured to insert the stopper member distally in the syringe interior relative to the syringe body and the needle hub, and
wherein the plunger member and the syringe body are configured such that, when the stopper member is inserted to the syringe body distal end, the needle proximal end pierces the stopper member to couple to the needle retention member and the needle retention member proximal end is disposed in the syringe body interior.

12. The system of claim 11, wherein the plunger member comprises
a plunger member body defining a plunger interior;
the needle retention member disposed in the plunger interior;
an energy-storage member disposed in the plunger interior and configured to withdraw the needle retention member proximally in the plunger interior; and
an energy-storage member latching member disposed in the plunger interior and having:
a latched state in which the energy-storage member latching member holds the energy-storage member in an energized state, and an
unlatched state in which the energy-storage member latching member does not restrain the energy-storage member and the energy-storage member transforms into a released state,
wherein when the stopper member is inserted to the syringe body distal end, the energy-storage member latching member is transformed from the latched state to the unlatched state, and the energy-storage member pulls needle proximally such that the sharp needle distal end is disposed in an interior of the needle hub.

13. A system for injection, comprising:
an autoinjector, comprising
a housing;
a syringe, comprising
a syringe body having a syringe body proximal end and a syringe body distal end, and defining a syringe body interior therebetween,
a needle hub assembly coupled to the syringe body at the syringe body distal end, and comprising
a needle hub, and
a needle removably coupled to the needle hub and having a sharp needle distal end and a needle proximal end feature,
a stopper member movably disposed in the syringe interior, and
a plunger member having a plunger member proximal end and a plunger member distal end, and coupled to the stopper member at the plunger member distal end,
wherein the plunger member is configured to insert the stopper member distally in the syringe interior relative to the syringe body and the needle hub, and
wherein the plunger member and the syringe body are configured such that, when the stopper member is inserted to the syringe body distal end, the plunger member proximal end is flush with the syringe body proximal end or disposed in the syringe body interior.

14. The system of claim 13, wherein the autoinjector comprises an activation button to initiate an injection using the syringe.

15. The system of claim 13, wherein the autoinjector comprises a display to communicate with a user.

16. The system of claim 13, wherein the autoinjector comprises a cover.

17. The system of claim 13, wherein the autoinjector defines a syringe nest configured to accept the syringe.

18. The system of claim 17, wherein the syringe nest is configured to be moved along a longitudinal axis of the autoinjector.

19. The system of claim 13, comprising a pushrod configured to advance the plunger member distally.

20. The system of claim 13, wherein the syringe is configured to retract the sharp needle distal end into the needle hub to render the syringe safe.
